(19) United States
(12) Patent Application Publication
(10) Pub. No.: US 2024/0002755 A1
(43) Pub. Date: Jan. 4, 2024

(54) FLEXIBLE, DISSOLVABLE, POROUS SHEETS

(71) Applicant: The Procter & Gamble Company, Cincinnati, OH (US)

(72) Inventors: Carl David MACNAMARA, Beijing (CN); Hongsing TAN, Beijing (CN)

(21) Appl. No.: 18/327,106

(22) Filed: Jun. 1, 2023

(30) Foreign Application Priority Data
Jul. 4, 2022 (WO) PCT/CN2022/103649

(51) Int. Cl. C11D 3/37; C11D 3/20; C11D 1/29; C11D 1/22; C11D 17/04

(52) U.S. Cl. CPC C11D 3/3753; C11D 3/2065; C11D 1/29; C11D 1/22; C11D 17/044

(57) ABSTRACT

This provides a flexible, dissolvable, and porous sheet that includes: a) from 50 wt % to 85 wt % of a water-soluble polymer; b) from 1 wt % to 40 wt % of a surfactant; and c) from 10 wt % to 40 wt % of glycerin, wherein said flexible, dissolvable, and porous sheet is characterized by a Percent Open Cell Content of from 80% to 99% and an Overall Average Pore Size of from 100 μm to 2000 μm.

### FIELD OF THE INVENTION

The present application relates to flexible, dissolvable, porous sheets with improved structural integrity.

### BACKGROUND OF THE INVENTION

Flexible and dissolvable detersive sheets comprising surfactant(s) and other active ingredients in a water-soluble polymeric carrier or matrix are well known. Such sheets are particularly useful for delivering surfactants and other active ingredients upon dissolution in water. In comparison with traditional granular or liquid detergents in the same product category, such sheets have better structural integrity, are more concentrated and easier to store, ship/transport, carry, and handle. In comparison with solid tablet detergents in the same product category, such sheets are more flexible and less brittle, with better sensory appeal to the consumers.

However, such flexible and dissolvable sheets may suffer from significantly slow dissolution in water, especially in comparison with the traditional granular or liquid product form.

To improve dissolution, WO2010077627, WO2012138820, WO2020147000 and WO2021102935 disclose various processes for forming porous sheets with open-celled foam (OCF) structures characterized by a Percent Open Cell Content of 80% or more. Although such OCF structures significantly improve the dissolution rate of the resulting sheets, they may adversely affect the tensile strength of such sheets. Correspondingly, the resulting sheets may have poorer structural integrity and are more suspectable to rapture under external forces.

It is therefore desirable to provide flexible, dissolvable, porous sheets with higher tensile strength and correspondingly improved structural integrity. Further, it will be advantageous to maintain satisfactory dissolution profile of such sheets.

### SUMMARY OF THE INVENTION

The present application provides a flexible, dissolvable, and porous sheet that comprises: a) from about 50% to about 85% of a water-soluble polymer by total weight of such sheet; b) from about 1% to about 40% of a surfactant by total weight of such sheet; and c) from about 10% to about 40% of glycerin by total weight of such sheet, wherein said flexible, dissolvable, and porous sheet is characterized by a Percent Open Cell Content of from about 80% to about 99% and an Overall Average Pore Size of from about 100 μm to about 2000 μm. Without being bound by any theory, it is believed that the relatively high level of water-soluble polymer in such sheet helps to improve its tensile strength, resulting in improved structural integrity and reduced risk of rapture under external forces, in comparison with similar sheets containing lower levels of water-soluble polymer. Further, it is also believed that the relatively high level of glycerin in such sheet helps to improve the dissolution profile and ensure fast dissolution in water, in comparison with similar sheets containing lower levels of glycerin.

Preferably, the flexible, dissolvable, and porous sheet as described hereinabove comprises from about 55% to about 80%, preferably from about 60% to about 75%, of said water-soluble polymer by total weight of said sheet. More preferably, said water-soluble polymer is selected from the group consisting of polyvinyl alcohols, polyvinylpyrrolidones, polyalkylene oxides, starch and starch derivatives, pullulan, gelatin, hydroxypropylmethylcelluloses, methycelluloses, carboxymethycelluloses, and any combinations thereof.

Still more preferably, said water-soluble polymer is a polyvinyl alcohol characterized by: (1) a weight average molecular weight of from about 50,000 to about 400,000 Daltons, more preferably from about 60,000 to about 300,000 Daltons, still more preferably from about 70,000 to about 200,000 Daltons, most preferably from about 80,000 to about 150,000 Daltons; and (2) a degree of hydrolysis ranging from about 60% to about 99%, preferably from about 70% to about 95%, more preferably from about 80% to about 90%. Most preferably, said water-soluble polymer is a blend of polyvinyl alcohols, which comprises the above-disclosed polyvinyl alcohol and an additional polyvinyl alcohol that is characterized by: (1) a weight average molecular weight of from about 5,000 to about 100,000 Daltons, more preferably from about 10,000 to about 50,000 Daltons, still more preferably from about 15,000 to about 40,000 Daltons, most preferably from about 20,000 to about 35,000 Daltons; and (2) a degree of hydrolysis ranging from about 60% to about 99%, preferably from about 70% to about 95%, more preferably from about 80% to about 90%. Preferably, the weight ratio of said additional polyvinyl alcohol to said polyvinyl alcohol ranges from about 0.1 to about 0.9, preferably from about 0.2 to about 0.8, more preferably from about 0.3 to about 0.7, most preferably from about 0.4 to about 0.6.

The above-disclosed flexible, dissolvable, and porous sheet preferably comprises from about 2% to about 30%, more preferably from about 5% to about 20%, most preferably from about 8% to about 15%, of the surfactant by total weight of said sheet. Such surfactant is preferably an anionic surfactant selected from the group consisting of C₆-C₂₀ linear alkylbenzene sulphonates (LAS), C₆-C₂₀ linear or branched alkylalkoxy sulfates (AAS), and any combinations thereof.

The above-disclosed flexible, dissolvable, and porous sheet preferably comprises from about 12% to about 30%, preferably from about 15% to about 25% of glycerin by total weight of said sheet.

Further, the flexible, dissolvable, and porous sheet of the present application may be characterized by any one or more of the following parameters:
a Percent Open Cell Content of from about 85% to about 99%, preferably from about 90% to about 99%; and/or
an Overall Average Pore Size of from about 150 μm to about 1000 μm, preferably from about 200 μm to about 600 μm; and/or
an Average Cell Wall Thickness of from about 5 μm to about 200 μm, preferably from about 10 μm to about 100 μm, more preferably from about 10 μm to about 80 μm; and/or
a final moisture content of from about 0.5% to about 25%, preferably from about 1% to about 20%, more preferably from about 3% to about 10%, by weight of said sheet; and/or
a thickness of from about 0.3 mm to about 4 mm, preferably from about 0.35 mm to about 3 mm, more preferably from about 0.4 mm to about 3 mm, still more preferably from about 0.45 mm to about 2 mm, most preferably from about 0.5 mm to about 1.5 mm; and/or
a basis weight of from about 15 grams/m² to about 1000 grams/m², preferably from about 20 grams/m² to about 700 grams/m², more preferably from about 30 grams/m² to about 300 grams/m², most preferably from about 35 grams/m² to about 200 grams/m²; and/or
a density of from about 0.05 grams/cm³ to about 0.5 grams/cm³, preferably from about 0.06 grams/cm³ to about 0.4 grams/cm³, more preferably from about 0.07 grams/cm³ to about 0.2 grams/cm³, most preferably from about 0.075 grams/cm³ to about 0.15 grams/cm³; and/or
a Specific Surface Area of from about 0.03 m²/g to about 0.25 m²/g, preferably from about 0.04 m²/g to about 0.22 m²/g, more preferably from about 0.05 m²/g to about 0.2 m²/g, most preferably from about 0.1 m²/g to about 0.18 m²/g.

The present application also provides a unitary detergent article that comprises: (1) two or more flexible, dissolvable, and porous sheets as mentioned hereinabove; and (2) one or more solid dissolvable components located between said two or more sheets, while each of such one or more solid dissolvable components comprises a cleansing active. The solid dissolvable components can be selected from the group consisting of particles, pastes, layers, films, sheets, and any combinations thereof. For example, such solid dissolvable components can be: (i) a plurality of discrete particles; and/or (ii) one or more continuous layers of paste; and/or (iii) one or more discontinuous layer of paste; and/or (iv) one or more fibrous sheets; and/or (v) one or more non-fibrous sheets. The cleaning active can be selected from the group consisting of fabric care actives, dishwashing actives, hard surface cleaning actives, beauty and/or skin care actives, personal cleansing actives, hair care actives, oral care actives, feminine care actives, baby care actives, and any combinations thereof.

These and other aspects of the present invention will become more apparent upon reading the following detailed description of the invention.

### DETAILED DESCRIPTION OF THE INVENTION

Features and benefits of the various embodiments of the present invention will become apparent from the following description, which includes examples of specific embodiments intended to give a broad representation of the invention. Various modifications will be apparent to those skilled in the art from this description and from practice of the invention. The scope of the present invention is not intended to be limited to the particular forms disclosed and the invention covers all modifications, equivalents, and alternatives falling within the spirit and scope of the invention as defined by the claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

As used herein, articles such as “a” and “an” when used in a claim, are understood to mean one or more of what is claimed or described. The terms “comprise,” “comprises,” “comprising,” “contain,” “contains,” “containing,” “include,” “includes” and “including” are all meant to be non-limiting.

As used herein, the terms “consisting essentially of” means that the composition contains no ingredient that will interfere with benefits or functions of those ingredients that are explicitly disclosed. Further, the term “substantially free of” or “substantially free from” means that the indicated material is present in the amount of from 0 wt % to about 5 wt %, preferably from 0 wt % to about 3 wt %, more preferably from 0 wt % to about 1 wt %. The term “essentially free of” or “essentially free from” means that the indicated material is at the very minimal not deliberately added to the composition or product, or preferably not present at an analytically detectible level in such composition or product. It may include compositions or products in which the indicated material is present only as an impurity of one or more of the materials deliberately added to such compositions or products.

The term “flexible” as used herein refers to the ability of an article to withstand stress without breakage or significant fracture when it is bent at about 900 along a center line perpendicular to its longitudinal direction. Preferably, such article can undergo significant elastic deformation and is characterized by a Young's Modulus of no more than about 5 GPa, preferably no more than about 1 GPa, more preferably no more than about 0.5 GPa, most preferably no more than about 0.2 GPa.

The term “dissolvable” as used herein refers to the ability of an article to completely or substantially dissolve in a sufficient amount of deionized water at 20° C. and under the atmospheric pressure within eight (8) hours without any stirring, leaving less than about 5 wt % undissolved residues.

The term “solid” as used herein refers to the ability of an article to substantially retain its shape (i.e., without any visible change in its shape) at 20° C. and under the atmospheric pressure, when it is not confined and when no external force is applied thereto.

The term “porous” as used herein refers to a solid structure containing voids or cells that are filled with a gas (such as air) or a fluid. The term “open celled foam” or “open cell pore structure” as used herein refers to a solid structure containing an interconnected network of such voids or cells, which do not collapse during the drying process, thereby maintaining the physical strength and cohesiveness of the solid as well as the interconnectivity of the voids/cells. The interconnectivity of the structure may be described by a Percent Open Cell Content (%), which is measured by Test 1 disclosed hereinafter.

The term “sheet” as used herein refers to a non-fibrous structure having a three-dimensional shape, i.e., with a thickness, a length, and a width, while the length-to-thickness aspect ratio and the width-to-thickness aspect ratio are both at least about 5:1, and the length-to-width ratio is at least about 1:1. Preferably, the length-to-thickness aspect ratio and the width-to-thickness aspect ratio are both at least about 10:1, more preferably at least about 15:1, most preferably at least about 20:1; and the length-to-width aspect ratio is preferably at least about 1.2:1, more preferably at least about 1.5:1, most preferably at least about 1.618:1.

The term “water-soluble” as used herein refers to the ability of a sample material to completely dissolve in or disperse into water leaving no visible solids or forming no visibly separate phase, when at least about 25 grams, preferably at least about 50 grams, more preferably at least about 100 grams, most preferably at least about 200 grams, of such material is placed in one liter (1 L) of deionized water at 20° C. and under the atmospheric pressure with sufficient stirring.

As used herein, the term “unitary” refers to a structure containing a plurality of distinctive parts that are combined together to form a visually coherent and structurally integral article.

As used herein, the term “discrete” refers to particles that are structurally distinctive from each other either under naked human eyes or under electronic imaging devices, such as scanning electron microscope (SEM) and transmission electron microscope (TEM). Preferably, the discrete particles of the present invention are structurally distinctive from each other under naked human eyes.

As used herein, the term “particle” refers to a solid matter of minute quantity, such as a powder, granule, encapsulate, microcapsule, and/or prill. The particles of the present invention can be spheres, rods, plates, tubes, squares, rectangles, discs, stars or flakes of regular or irregular shapes, but they are non-fibrous. The particles of the present invention may have a median particle size of 2000 μm or less. Preferably, the particles of the present invention have a median particle size ranging from about 1 μm to about 2000 μm, more preferably from about 10 μm to about 1800 μm, still more preferably from about 50 μm to about 1700 μm, still more preferably from about 100 μm to about 1500 μm, still more preferably from about 250 μm to about 1000 μm, most preferably from about 300 μm to about 800 μm.

As used herein, the term “non-fibrous” refers to a structure that is free of or substantially free of fibrous elements. “Fibrous element” and “filaments” are used interchangeably here to refer to elongated particles having a length greatly exceeding its average cross-sectional diameter, i.e., a length-to-diameter aspect ratio of at about least 10:1, and preferably such elongated particles have an average cross-sectional diameter of no more than about 1 mm.

As used herein, all concentrations and ratios are on a weight basis unless otherwise specified. All temperatures herein are in degrees Celsius (° C.) unless otherwise indicated. All conditions herein are at 20° C. and under the atmospheric pressure, unless otherwise specifically stated. All polymer molecular weights are determined by weight average number molecular weight unless otherwise specifically noted.

### Water-Soluble Polymer

The present invention provides flexible, dissolvable, and porous sheets that are formed by the same or similar processes as those disclosed in WO2020147000 and WO2021102935, and such sheets are characterized by the same open cell foam (OCF) structures and physical properties as those disclosed in WO2020147000 and WO2021102935, but with a significantly higher level of the water-soluble polymer (i.e., 50-85 wt % in comparison with 5-40 wt %). Without being bound by any theory, it is believed that the relatively high level of water-soluble polymer in the sheets of the present invention helps to improve the tensile strength, resulting in improved structural integrity and reduced risk of rapture under external forces, in comparison with similar sheets containing lower levels of water-soluble polymer disclosed by WO2020147000 and WO2021102935.

Preferably, the flexible, dissolvable, and porous sheet of the present invention comprises from about 50% to about 85%, preferably from about 55% to about 80%, more preferably from about 60% to about 75% of said water-soluble polymer by total weight of the sheet.

Water-soluble polymers suitable for the practice of the present invention may be selected those with weight average molecular weights ranging from about 5,000 to about 400,000 Daltons, more preferably from about 10,000 to about 300,000 Daltons, still more preferably from about 15,000 to about 200,000 Daltons, most preferably from about 20,000 to about 150,000 Daltons. The weight average molecular weight is computed by summing the average molecular weights of each polymer raw material multiplied by their respective relative weight percentages by weight of the total weight of polymers present within the porous solid. The weight average molecular weight of the water-soluble polymer used herein may impact the viscosity of the wet pre-mixture, which may in turn influence the bubble number and size during the aeration step as well as the pore expansion/opening results during the drying step. Further, the weight average molecular weight of the water-soluble polymer may affect the overall film-forming properties of the wet pre-mixture and its compatibility/incompatibility with certain surfactants.

The water-soluble polymers of the present invention may also be selected from naturally sourced polymers including those of plant origin examples of which include karaya gum, tragacanth gum, gum Arabic, acemannan, konjac mannan, acacia gum, gum ghatti, whey protein isolate, and soy protein isolate; seed extracts including guar gum, locust bean gum, quince seed, and psyllium seed; seaweed extracts such as Carrageenan, alginates, and agar; fruit extracts (pectins); those of microbial origin including xanthan gum, gellan gum, pullulan, hyaluronic acid, chondroitin sulfate, and dextran; and those of animal origin including casein, gelatin, keratin, keratin hydrolysates, sulfonic keratins, albumin, collagen, glutelin, glucagons, gluten, zein, and shellac.

Modified natural polymers can also be used as water-soluble polymers in the present invention. Suitable modified natural polymers include, but are not limited to, cellulose derivatives such as hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose and other cellulose ethers/esters; and guar derivatives such as hydroxypropyl guar.

The water-soluble polymer of the present invention may include starch. As used herein, the term “starch” includes both naturally occurring and modified starches. Typical natural sources for starches can include cereals, tubers, roots, legumes and fruits. More specific natural sources can include corn, pea, potato, banana, barley, wheat, rice, sago, amaranth, tapioca, arrowroot, canna, sorghum, and waxy or high amylase varieties thereof. The natural starches can be modified by any modification method known in the art to form modified starches, including physically modified starches, such as sheared starches or thermally-inhibited starches; chemically modified starches, such as those which have been cross-linked, acetylated, and organically esterified, hydroxyethylated, and hydroxypropylated, phosphorylated, and inorganically esterified, cationic, anionic, nonionic, amphoteric and zwitterionic, and succinate and substituted succinate derivatives thereof; conversion products derived from any of the starches, including fluidity or thin-boiling starches prepared by oxidation, enzyme conversion, acid hydrolysis, heat or acid dextrinization, thermal and or sheared products may also be useful herein; and pregelatinized starches which are known in the art.

The water-soluble polymers of the present invention may include, but are not limited to, synthetic polymers including polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, copolymers of acrylic acid and methyl acrylate, polyurethanes, polycarboxylic acids, polyvinyl acetates, polyesters, polyamides, polyamines, polyethyleneimines, maleic/(acrylate or methacrylate) copolymers, copolymers of methylvinyl ether and of maleic anhydride, copolymers of vinyl acetate and crotonic acid, copolymers of vinylpyrrolidone and of vinyl acetate, copolymers of vinylpyrrolidone and of caprolactam, vinyl pyrollidone/vinyl acetate copolymers, copolymers of anionic, cationic and amphoteric monomers, and combinations thereof.

Preferred water-soluble polymers of the present invention include are selected from the group consisting of polyvinyl alcohols, polyvinylpyrrolidones, polyalkylene oxides, starch and starch derivatives, pullulan, gelatin, hydroxypropylmethylcelluloses, methycelluloses, carboxymethycelluloses, and any combinations thereof. More preferred water-soluble polymers of the present invention include polyvinyl alcohols, and hydroxypropylmethylcelluloses.

Most preferably, the water-soluble polymer used in the present invention is a polyvinyl alcohol or a blend of polyvinyl alcohols. Suitable polyvinyl alcohols for use in the present invention are preferably characterized by a degree of hydrolysis ranging from about 40% to about 100%, preferably from about 50% to about 95%, more preferably from about 65% to about 92%, most preferably from about 70% to about 90%. Commercially available polyvinyl alcohols include those from Celanese Corporation (Texas, USA) under the CELVOL trade name including, but not limited to, CELVOL 523, CELVOL 530, CELVOL 540, CELVOL 518, CELVOL 513, CELVOL 508, CELVOL 504; those from Kuraray Europe GmbH (Frankfurt, Germany) under the Mowiol® and POVAL™ trade names; and PVA 1788 (also referred to as PVA BP17) commercially available from various suppliers including Lubon Vinylon Co. (Nanjing, China); and combinations thereof.

In a particularly preferred embodiment of the present invention, the flexible, porous, dissolvable sheet comprises from about 50% to about 85%, more preferably from about 60% to about 80%, most preferably from about 65% to about 75%, by total weight of such sheet, of a polyvinyl alcohol having: (1) a weight average molecular weight ranging from about 50,000 to about 400,000 Daltons, preferably from about 60,000 to about 300,000 Daltons, more preferably from about 70,000 to about 200,000 Daltons, most preferably from about 80,000 to about 150,000 Daltons; and (2) a degree of hydrolysis ranging from about 60% to about 99%, preferably from about 70% to about 95%, more preferably from about 80% to about 90%.

In another particularly preferred embodiment of the present invention, the flexible, porous, dissolvable sheet comprises from about 50% to about 85%, more preferably from about 60% to about 80%, most preferably from about 65% to about 75%, by total weight of such sheet, of a blend of polyvinyl alcohols that comprise the above-disclosed polyvinyl alcohol (i.e., a first PVA) and an additional polyvinyl alcohol (i.e., a second PVA) that is characterized by: (1) a weight average molecular weight of from about 5,000 to about 100,000 Daltons, more preferably from about 10,000 to about 50,000 Daltons, still more preferably from about 15,000 to about 40,000 Daltons, most preferably from about 20,000 to about 35,000 Daltons; and (2) a degree of hydrolysis ranging from about 60% to about 99%, preferably from about 70% to about 95%, more preferably from about 80% to about 90%. The weight ratio of the second PVA (with lower Mw) over the first PVA (with higher Mw) may range from about 0.1 to about 0.9, preferably from about 0.2 to about 0.8, more preferably from about 0.3 to about 0.7, most preferably from about 0.4 to about 0.6. Without being bound any theory, it is believed that such a PVA blend containing a relatively low Mw PVA and a relatively high Mw PVA at a weight ratio between about 0.1 and about 0.9 provides sheets with better solubility, higher tensile strength, and correspondingly improved processability.

In addition to polyvinyl alcohols as mentioned hereinabove, a single starch or a combination of starches may be used as a filler material in such an amount as to reduce the overall level of water-soluble polymers required, so long as it helps provide the flexible, dissolvable, and porous sheets with the requisite structure and physical/chemical characteristics as described herein. However, too much starch may comprise the solubility and structural integrity of the sheets. Therefore, in preferred embodiments of the present invention, it is desired that the sheet comprises no more than about 20%, preferably from 0% to about 10%, more preferably from 0% to about 5%, most preferably from 0% to about 1%, by weight of said sheet, of starch.

### Surfactants

In addition to the water-soluble polymer described hereinabove, the flexible, dissolvable, and porous sheet of the present invention comprises one or more surfactants in the amount ranging from about 1% to about 40%, preferably from about 2% to about 30%, more preferably from about 5% to about 20%, most preferably from about 8% to about 15%, by total weight of such sheet. The surfactants may function as emulsifying agents during the aeration process to create a sufficient amount of stable bubbles for forming the desired OCF structure of the present invention. Further, the surfactants may function as active ingredients for delivering a desired cleansing benefit.

In a preferred embodiment of the present invention, the flexible, dissolvable, and porous sheet comprises one or more surfactants selected from the group consisting of anionic surfactants, nonionic surfactants, cationic surfactants, zwitterionic surfactants, amphoteric surfactants, polymeric surfactants or combinations thereof. Depending on the desired application of such sheet and the desired consumer benefit to be achieved, different surfactants can be selected.

The surfactant as used herein may include both surfactants from the conventional sense (i.e., those providing a consumer-noticeable lathering effect) and emulsifiers (i.e., those that do not provide any lathering performance but are intended primarily as a process aid in making a stable foam structure). Examples of emulsifiers for use as a surfactant component herein include mono- and di-glycerides, fatty alcohols, polyglycerol esters, propylene glycol esters, sorbitan esters and other emulsifiers known or otherwise commonly used to stabilize air interfaces.

Non-limiting examples of anionic surfactants suitable for use herein include alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

One category of anionic surfactants particularly suitable for practice of the present invention include C₆-C₂₀ linear alkylbenzene sulphonate (LAS) surfactant. LAS surfactants are well known in the art and can be readily obtained by sulfonating commercially available linear alkylbenzenes. Exemplary C₁₀-C₂₀ linear alkylbenzene sulfonates that can be used in the present invention include alkali metal, alkaline earth metal or ammonium salts of C₁₀-C₂₀ linear alkylbenzene sulfonic acids, and preferably the sodium, potassium, magnesium and/or ammonium salts of C₁₁-C₈₅ or C₁₁-C₁₄ linear alkylbenzene sulfonic acids. More preferred are the sodium or potassium salts of C₁₂ and/or C₁₄ linear alkylbenzene sulfonic acids, and most preferred is the sodium salt of C₁₂ and/or C₁₄ linear alkylbenzene sulfonic acid, i.e., sodium dodecylbenzene sulfonate or sodium tetradecylbenzene sulfonate.

LAS provides superior cleaning benefit and is especially suitable for use in laundry detergent applications. It has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, LAS can be used as a major surfactant, i.e., present in an amount that is more than 50% by weight of the total surfactant content in the sheet, without adversely affecting the film-forming performance and stability of the overall composition. If present, the amount of LAS in the sheet of the present invention may range from about 1% to about 40%, preferably from about 2% to about 30%, more preferably from about 5% to about 20%, most preferably from about 8% to about 15%, by total weight of the sheet.

Another category of anionic surfactants suitable for practice of the present invention include sodium trideceth sulfates (STS) having a weight average degree of alkoxylation ranging from about 0.5 to about 5, preferably from about 0.8 to about 4, more preferably from about 1 to about 3, most preferably from about 1.5 to about 2.5. Trideceth is a 13-carbon branched alkoxylated hydrocarbon comprising, in one embodiment, an average of at least 1 methyl branch per molecule. STS used by the present invention may be include ST(EOxPOy)S, while EOx refers to repeating ethylene oxide units with a repeating number x ranging from 0 to 5, preferably from 1 to 4, more preferably from 1 to 3, and while POy refers to repeating propylene oxide units with a repeating number y ranging from 0 to 5, preferably from 0 to 4, more preferably from 0 to 2. It is understood that a material such as ST2S with a weight average degree of ethoxylation of about 2, for example, may comprise a significant amount of molecules which have no ethoxylate, 1 mole ethoxylate, 3 mole ethoxylate, and so on, while the distribution of ethoxylation can be broad, narrow or truncated, which still results in an overall weight average degree of ethoxylation of about 2. STS is particularly suitable for personal cleansing applications, and it has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, STS can be used as a major surfactant, i.e., present in an amount that is more than 50% by weight of the total surfactant content in the sheet, without adversely affecting the film-forming performance and stability of the overall composition. If present, the amount of STS in the sheet of the present invention may range from about 1% to about 40%, preferably from about 2% to about 30%, more preferably from about 5% to about 20%, most preferably from about 8% to about 15%, by total weight of the sheet.

Another category of anionic surfactants suitable for practice of the present invention include C₆-C₂₀ linear or branched alkylalkoxy sulfates (AAS). Among this category, linear or branched alkylethoxy sulfates (AES) having the respective formulae RO(C₂H₄O)ₓSO₃M are particularly preferred, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms. The AES surfactants are typically made as condensation products of ethylene oxide and monohydric alcohol's having from about 6 to about 20 carbon atoms. Useful alcohols can be derived from fats, e.g., coconut oil or tallow, or can be synthetic. Lauryl alcohol and straight chain alcohol's derived from coconut oil are preferred herein. Such alcohol's are reacted with about 1 to about 10, preferably from about 3 to about 5, and especially about 3, molar proportions of ethylene oxide and the resulting mixture of molecular species having, for example, an average of 3 moles of ethylene oxide per mole of alcohol, is sulfated and neutralized. Highly preferred AES are those comprising a mixture of individual compounds, said mixture having an average alkyl chain length of from about 10 to about 16 carbon atoms and an average degree of ethoxylation of from about 1 to about 4 moles of ethylene oxide. If present, the the amount of AAS in the sheet of the present invention may range from about 1% to about 40%, preferably from about 2% to about 30%, more preferably from about 5% to about 20%, most preferably from about 8% to about 15%, by total weight of the solid sheet article.

Another category of anionic surfactants suitable for practice of the present invention include alkyl sulfates. These materials have the respective formulae ROSO₃M, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 6 to about 20, preferably about 10 to about 18, carbon atoms; and M is a cation. Preferred are alkali metal and ammonium sulfonated C₁₀₋₁₈ n-paraffins. Other suitable anionic surfactants include olefin sulfonates having about 12 to about 24 carbon atoms. The α-olefins from which the olefin sulfonates are derived are mono-olefins having about 12 to about 24 carbon atoms, preferably about 14 to about 16 carbon atoms. Preferably, they are straight chain olefins.

Another class of anionic surfactants suitable for use in the fabric and home care compositions is the β-alkyloxy alkane sulfonates. These compounds have the following formula:

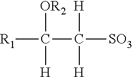

where R₁ is a straight chain alkyl group having from about 6 to about 20 carbon atoms, R₂ is a lower alkyl group having from about 1 (preferred) to about 3 carbon atoms, and M is a water-soluble cation as hereinbefore described.

Additional examples of suitable anionic surfactants are the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Still other suitable anionic surfactants are the succinamates, examples of which include disodium N-octadecylsulfosuccinamate; diammoniumlauryl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; and dioctyl esters of sodium sulfosuccinic acid.

Nonionic surfactants that can be included into the solid sheet article of the present invention may be any conventional nonionic surfactants, including but not limited to: alkyl alkoxylated alcohols, alkyl alkoxylated phenols, alkyl polysaccharides (especially alkyl glucosides and alkyl polyglucosides), polyhydroxy fatty acid amides, alkoxylated fatty acid esters, sucrose esters, sorbitan esters and alkoxylated derivatives of sorbitan esters, amine oxides, and the like. Preferred nonionic surfactants are those of the formula R¹(OC₂H₄)ₙOH, wherein R¹ is a C₈-C₁₈ alkyl group or alkyl phenyl group, and n is from about 1 to about 80. Particularly preferred are C₈-C₁₈ alkyl ethoxylated alcohols having a weight average degree of ethoxylation from about 1 to about 20, preferably from about 5 to about 15, more preferably from about 7 to about 10, such as NEODOL® nonionic surfactants commercially available from Shell. Other non-limiting examples of nonionic surfactants useful herein include: C₆-C₁₂ alkyl phenol alkoxylates where the alkoxylate units may be ethyleneoxy units, propyleneoxy units, or a mixture thereof; C₁₂-C₁₈ alcohol and C₆-C₁₂ alkyl phenol condensates with ethylene oxide/propylene oxide block polymers such as Pluronic© from BASF; C₁₄-C₂₂ mid-chain branched alcohols (BA); C₁₄-C₂₂ mid-chain branched alkyl alkoxylates, BAEN, wherein x is from 1 to 30; alkyl polysaccharides, specifically alkyl polyglycosides; Polyhydroxy fatty acid amides; and ether capped poly(oxyalkylated) alcohol surfactants. Suitable nonionic surfactants also include those sold under the tradename Lutensol® from BASF.

In a preferred embodiment, the nonionic surfactant is selected from sorbitan esters and alkoxylated derivatives of sorbitan esters including sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), sorbitan isostearate, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), all available from Uniqema, and combinations thereof.

The most preferred nonionic surfactants for practice of the present invention include C₆-C₂₀ linear or branched alkylalkoxylated alcohols (AA) having a weight average degree of alkoxylation ranging from 5 to 15, more preferably C₁-C₁₄ linear ethoxylated alcohols having a weight average degree of alkoxylation ranging from 7 to 9. If present, the amount of AA-type nonionic surfactant(s) in the sheet of the present invention may range from about 1% to about 40%, preferably from about 2% to about 30%, more preferably from about 5% to about 20%, most preferably from 8% to 15%, by total weight of the sheet.

Amphoteric surfactants suitable for use in the sheet of the present invention includes those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate, and N-higher alkyl aspartic acids.

One category of amphoteric surfactants particularly suitable for incorporation into sheets with personal care applications (e.g., shampoo, facial or body cleanser, and the like) include alkylamphoacetates, such as lauroamphoacetate and cocoamphoacetate. Alkylamphoacetates can be comprised of monoacetates and diacetates. In some types of alkylamphoacetates, diacetates are impurities or unintended reaction products. If present, the amount of alkylamphoacetate(s) in the sheet of the present invention may range from about 1% to about 40%, preferably from about 2% to about 30%, more preferably from about 5% to about 20%, most preferably from about 8% to about 15%, by total weight of the sheet.

Zwitterionic surfactants suitable include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

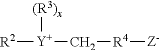

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use herein include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical, wherein R is a C₁₁-C₁₇ alkyl, is attached to the nitrogen atom of the betaine are also useful in this invention.

Cationic surfactants can also be utilized in the present invention, especially in fabric softener and hair conditioner products. When used in making products that contain cationic surfactants as the major surfactants, it is preferred that such cationic surfactants are present in an amount ranging from about 1% to about 40%, preferably from about 2% to about 30%, more preferably from about 5% to about 20%, most preferably from about 8% to about 15%, by total weight of the sheet.

Cationic surfactants may include DEQA compounds, which encompass a description of diamido actives as well as actives with mixed amido and ester linkages. Preferred DEQA compounds are typically made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyl-oxyethyl)-N,N-methylhydroxyethylammonium methylsulfate wherein the acyl group is derived from animal fats, unsaturated, and polyunsaturated, fatty acids.

Other suitable actives for use as a cationic surfactant include reaction products of fatty acids with dialkylenetriamines in, e.g., a molecular ratio of about 2:1, said reaction products containing compounds of the formula:

R¹—C(O)—NH—R²—NH—R³—NH—C(O)—R¹

wherein R¹, R² are defined as above, and each R³ is a C₁₋₆ alkylene group, preferably an ethylene group. Examples of these actives are reaction products of tallow acid, canola acid, or oleic acids with diethylenetriamine in a molecular ratio of about 2:1, said reaction product mixture containing N,N″-ditallowoyldiethylenetriamine, N,N″-dicanola-oyldiethylenetriamine, or N,N″-dioleoyldiethylenetriamine, respectively, with the formula:

R¹—C(O)—NH—CH₂CH₂—NH—CH₂CH₂—NH—C(O)—R¹

wherein R² and R³ are divalent ethylene groups, R¹ is defined above and an acceptable examples of this structure when R¹ is the oleoyl group of a commercially available oleic acid derived from a vegetable or animal source, include EMERSOL® 223LL or EMERSOL® 7021, available from Henkel Corporation.

Another active for use as a cationic surfactant has the formula:

[R¹—C(O)—NR—R²—N(R)₂—R³—NR—C(O)—R¹]⁺X⁻

wherein R, R¹, R², R³ and X⁻ are defined as above. Examples of this active are the di-fatty amidoamines based softener having the formula:

[R¹—C(O)—NH—CH₂CH₂—N(CH₃)(CH₂CH₂OH)—CH₂CH₂—NH—C(O)—R₁]⁺CH₃SO₄⁻

wherein R¹—C(O) is an oleoyl group, soft tallow group, or a hardened tallow group available commercially from Degussa under the trade names VARISOFT® 222LT, VARISOFT® 222, and VARISOFT® 110, respectively.

A second type of DEQA (“DEQA (2)”) compound suitable as a active for use as a cationic surfactant has the general formula:

[R₃N⁺CH₂CH(YR¹)(CH₂YR¹)]X⁻

wherein each Y, R, R¹, and X⁻ have the same meanings as before. An example of a preferred DEQA (2) is the “propyl” ester quaternary ammonium fabric softener active having the formula 1,2-di(acyloxy)-3-trimethylammoniopropane chloride.

Suitable polymeric surfactants for use in the sheets of the present invention include, but are not limited to, block copolymers of ethylene oxide and fatty alkyl residues, block copolymers of ethylene oxide and propylene oxide, hydrophobically modified polyacrylates, hydrophobically modified celluloses, silicone polyethers, silicone copolyol esters, diquaternary polydimethylsiloxanes, and co-modified amino/polyether silicones.

### Glycerin

In addition to the above-mentioned high level of water-soluble polymer (i.e., from about 50 wt % to about 85 wt %), the flexible, dissolvable, and porous sheet of the present invention is also characterized by a uniquely high level of glycerin, i.e., from about 10% to about 40%, preferably from about 12% to about 30%, more preferably from about 15% to about 25%, by total weight of such sheet.

In contrast, WO2019007954 discloses thin water-soluble sheets containing 65%-76.3% PVA and 17.7%-18% surfactants, but with only about 2.2% of glycerin (see Examples 1 and 3). Without being bound by any theory, it is believed that the relatively high level of glycerin in the flexible, dissolvable, porous sheets of the present invention helps to improve the dissolution profile and ensure fast dissolution in water, in comparison with similar sheets containing lower levels of glycerin.

### Additional Ingredients

In addition to the above-described ingredients (e.g., the water-soluble polymer, the surfactant(s), and glycerin), the flexible, dissolvable, and porous sheets of the present invention may comprise one or more additional ingredients, depending on its intended application. Such one or more additional ingredients may be selected from the group consisting of fabric care actives, dishwashing actives, hard surface cleaning actives, beauty and/or skin care actives, personal cleansing actives, hair care actives, oral care actives, feminine care actives, baby care actives, and any combinations thereof.

Suitable fabric care actives include but are not limited to: organic solvents (linear or branched lower C₁-C₈ alcohols, diols, glycerols or glycols; lower amine solvents such as C₁-C₄ alkanolamines, and mixtures thereof; more specifically 1,2-propanediol, ethanol, glycerol, monoethanolamine and triethanolamine), carriers, hydrotropes, builders, chelants, dispersants, enzymes and enzyme stabilizers, catalytic materials, bleaches (including photobleaches) and bleach activators, perfumes (including encapsulated perfumes or perfume microcapsules), colorants (such as pigments and dyes, including hueing dyes), brighteners, dye transfer inhibiting agents, clay soil removal/anti-redeposition agents, structurants, rheology modifiers, suds suppressors, processing aids, fabric softeners, anti-microbial agents, and the like.

Suitable hair care actives include but are not limited to: moisture control materials of class II for frizz reduction (salicylic acids and derivatives, organic alcohols, and esters), cationic surfactants (especially the water-insoluble type having a solubility in water at 25° C. of preferably below 0.5 g/100 g of water, more preferably below 0.3 g/100 g of water), high melting point fatty compounds (e.g., fatty alcohols, fatty acids, and mixtures thereof with a melting point of 25° C. or higher, preferably 40° C. or higher, more preferably 45° C. or higher, still more preferably 50° C. or higher), silicone compounds, conditioning agents (such as hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients), preservatives (such as benzyl alcohol, methyl paraben, propyl paraben and imidazolidinyl urea), pH adjusting agents (such as citric acid, sodium citrate, succinic acid, phosphoric acid, sodium hydroxide, sodium carbonate), salts (such as potassium acetate and sodium chloride), coloring agents, perfumes or fragrances, sequestering agents (such as disodium ethylenediamine tetra-acetate), ultraviolet and infrared screening and absorbing agents (such as octyl salicylate), hair bleaching agents, hair perming agents, hair fixatives, anti-dandruff agents, anti-microbial agents, hair growth or restorer agents, co-solvents or other additional solvents, and the like.

Suitable beauty and/or skin care actives include those materials approved for use in cosmetics and that are described in reference books such as the CTFA Cosmetic Ingredient Handbook, Second Edition, The Cosmetic, Toiletries, and Fragrance Association, Inc. 1988, 1992. Further non-limiting examples of suitable beauty and/or skin care actives include preservatives, perfumes or fragrances, coloring agents or dyes, thickeners, moisturizers, emollients, pharmaceutical actives, vitamins or nutrients, sunscreens, deodorants, sensates, plant extracts, nutrients, astringents, cosmetic particles, absorbent particles, fibers, anti-inflammatory agents, skin lightening agents, skin tone agent (which functions to improve the overall skin tone, and may include vitamin B3 compounds, sugar amines, hexamidine compounds, salicylic acid, 1,3-dihydroxy-4-alkybenzene such as hexylresorcinol and retinoids), skin tanning agents, exfoliating agents, humectants, enzymes, antioxidants, free radical scavengers, anti-wrinkle actives, anti-acne agents, acids, bases, minerals, suspending agents, pH modifiers, pigment particles, anti-microbial agents, insect repellents, shaving lotion agents, co-solvents or other additional solvents, and the like.

The flexible, dissolvable, and porous sheets of the present invention may further comprise other optional ingredients that are known for use or otherwise useful in compositions, provided that such optional materials are compatible with the selected essential materials described herein, or do not otherwise unduly impair product performance.

### Product Form

Non-limiting examples of products that can be formed by the flexible, dissolvable, and porous sheets of the present invention include laundry detergent products, fabric softening products, hand cleansing products, hair shampoo or other hair treatment products, body cleansing products, shaving preparation products, dish cleaning products, personal care products, moisturizing products, sunscreen products, beauty or skin care products, deodorizing products, oral care products, feminine cleansing products, baby care products, fragrance-containing products, and so forth.

For example, the flexible, dissolvable, and porous sheets of the present invention can be used to form a unitary detergent article comprising: (1) two or more of such sheets as mentioned hereinabove; and (2) one or more solid dissolvable components located between said two or more sheets, while each of said one or more solid dissolvable components comprises a cleansing active, e.g., selected from the group consisting of fabric care actives, dishwashing actives, hard surface cleaning actives, beauty and/or skin care actives, personal cleansing actives, hair care actives, oral care actives, feminine care actives, baby care actives, and any combinations thereof. The one or more solid dissolvable components can be particles, pastes, layers, films, sheets, and any combinations thereof.

The flexible, dissolvable, and porous sheets of the present invention characterized by its improved structural integrity and better resistance to external forces as well as a good dissolution profile with fast dissolution rate in water, are particularly suitable for use as dissolvable packaging layers or protective outer layers to enclose solid dissolvable components with higher active contents and stronger cleansing performance, but poorer structural integrity. Such use of the flexible, dissolvable, and porous sheets of the present invention provides a more sustainable solution for product packaging and design that may result in significantly reduced plastic waste.

In a preferred but non-limiting example, the solid dissolvable components are a plurality of discrete particles, which are sandwiched between two or more flexible, dissolvable, and porous sheets. More preferably, the two or more sheets are then sealed along their peripheries to prevent leakage of the discrete particles therefrom, e.g., by heating, pressing and/or cutting to form edge seals.

It is preferred that such discrete particles have a relatively low water/moisture content (e.g., no more than about 10 wt % of total water/moisture, preferably no more than about 8 wt % of total water/moisture, more preferably no more than about 5 wt % of total moisture), especially a relatively low free/unbound water content (e.g., no more than about 3 wt % of free or unbound water, preferably no more than about 1 wt % of free or unbound water), so that water from such discrete particles will not compromise structural integrity of adjacent sheets. Further, the controlled moisture content in such discrete particles reduces the risk of gelling in the particles themselves. Discrete particles suitable for use in the present invention can be any shapes selected from the group consisting of spheres, rods, plates, tubes, squares, rectangles, discs, stars, flakes of regular or irregular shapes, and combinations thereof, as long as they are non-fibrous. They may have a median particle size of 2000 μm or less, Preferably, such discrete particles have a median particle size ranging from about 1 μm to about 2000 μm, preferably from about 10 μm to about 1800 μm, more preferably from about 50 μm to about 1700 μm, still more preferably from about 100 μm to about 1500 μm, still more preferably from about 250 μm to about 1000 μm, most preferably from about 300 μm to about 800 μm. The bulk density of such discrete particles may range from 500 g/L to 1000 g/L, preferably from 600 g/L to 900 g/L, more preferably from 700 g/L to 800 g/L.

In another preferred but non-limiting example of the present invention, the solid dissolvable components are one or more continuous or discontinuous layers of paste, which may be formed by, for example, a non-aqueous liquid carrier, a plurality of solid particles and optionally a thickening agent, as disclosed by WO2021102935.

In still another preferred but non-limiting example of the present invention, the solid dissolvable components are one or more one or more fibrous sheets as those disclosed in WO2018137709 and WO2018140668.

In yet another preferred but non-limiting example of the present invention, the solid dissolvable components are one or more non-fibrous sheets, and preferably one or more flexible, dissolvable and porous sheets as those disclosed in WO2010077627, WO2012138820, WO2020147000 and WO2021102935.

The solid dissolvable components of the present invention can be characterized by a relatively high level of cleansing active (e.g., surfactants, polymers, enzymes, etc.) in comparison with that in the flexible, dissolvable, and porous sheets. For example, the cleansing active(s) can be present at least 30%, preferably at least 50%, more preferably at least 60%, and most preferably at least 70%, by total weight of such discrete particles. Such cleansing active can be selected from the group consisting of fabric care actives, dishwashing actives, hard surface cleaning actives, beauty and/or skin care actives, personal cleansing actives, hair care actives, oral care actives, feminine care actives, baby care actives, and any combinations thereof.

The solid dissolvable components of the present invention may optionally include one or more detergent ingredients for assisting or enhancing cleaning performance or to modify the aesthetics thereof. Illustrative examples of such detergent ingredients include: (1) surfactants, such as anionic surfactants, nonionic surfactants, cationic surfactants, amphoteric surfactants, and zwitterionic surfactants as mentioned hereinabove; (2) inorganic and/or organic builders, such as carbonates (including bicarbonates and sesquicarbonates), sulphates, phosphates (exemplified by the tripolyphosphates, pyrophosphates, and glassy polymeric meta-phosphates), phosphonates, phytic acid, silicates, zeolite, citrates, polycarboxylates and salts thereof (such as mellitic acid, succinic acid, oxydisuccinic acid, polymaleic acid, benzene 1,3,5-tricarboxylic acid, carboxymethyloxysuccinic acid, and soluble salts thereof), ether hydroxypolycarboxylates, copolymers of maleic anhydride with ethylene or vinyl methyl ether, 1,3,5-trihydroxy benzene-2,4,6-trisulphonic acid, 3,3-dicarboxy-4-oxa-1,6-hexanedioates, polyacetic acids (such as ethylenediamine tetraacetic acid and nitrilotriacetic acid) and salts thereof, fatty acids (such as C₁₂-C₁₈ monocarboxylic acids); (3) chelating agents, such as iron and/or manganese-chelating agents selected from the group consisting of amino carboxylates, amino phosphonates, polyfunctionally-substituted aromatic chelating agents and mixtures therein; (4) clay soil removal/anti-redeposition agents, such as water-soluble ethoxylated amines (particularly ethoxylated tetraethylene-pentamine); (5) polymeric dispersing agents, such as polymeric polycarboxylates, acrylic/maleic-based copolymers and water-soluble salts thereof of, hydroxypropylacrylate, maleic/acrylic/vinyl alcohol terpolymers, polyaspartates and polyglutamates; (6) optical brighteners, which include but are not limited to derivatives of stilbene, pyrazoline, coumarin, carboxylic acid, methinecyanines, dibenzothiphene-5,5-dioxide, azoles, 5- and 6-membered-ring heterocycles, and the like; (7) suds suppressors, such as monocarboxylic fatty acids and soluble salts thereof, high molecular weight hydrocarbons (e.g., paraffins, haloparaffins, fatty acid esters, fatty acid esters of monovalent alcohols, aliphatic C₁₈-C₄₀ ketones, etc.), N-alkylated amino triazines, propylene oxide, monostearyl phosphates, silicones or derivatives thereof, secondary alcohols (e.g., 2-alkyl alkanols) and mixtures of such alcohols with silicone oils; (8) suds boosters, such as C₁₀-C₁₆ alkanolamides, C₁₀-C₁₄ monoethanol and diethanol amides, high sudsing surfactants (e.g., amine oxides, betaines and sultaines), and soluble magnesium salts (e.g., MgCl₂, MgSO₄, and the like); (9) fabric softeners, such as smectite clays, amine softeners and cationic softeners; (10) dye transfer inhibiting agents, such as polyvinyl pyrrolidone polymers, polyamine N-oxide polymers, copolymers of N-vinylpyrrolidone and N-vinylimidazole, manganese phthalocyanine, peroxidases, and mixtures thereof; (11) enzymes, such as proteases, amylases, lipases, cellulases, and peroxidases, and mixtures thereof; (12) enzyme stabilizers, which include water-soluble sources of calcium and/or magnesium ions, boric acid or borates (such as boric oxide, borax and other alkali metal borates); (13) bleaching agents, such as percarbonates (e.g., sodium carbonate peroxyhydrate, sodium pyrophosphate peroxyhydrate, urea peroxyhydrate, and sodium peroxide), persulfates, perborates, magnesium monoperoxyphthalate hexahydrate, the magnesium salt of metachloro perbenzoic acid, 4-nonylamino-4-oxoperoxybutyric acid and diperoxydodecanedioic acid, 6-nonylamino-6-oxoperoxycaproic acid, and photoactivated bleaching agents (e.g., sulfonated zinc and/or aluminum phthalocyanines); (14) bleach activators, such as nonanoyloxybenzene sulfonate (NOBS), tetraacetyl ethylene diamine (TAED), amido-derived bleach activators including (6-octanamidocaproyl)oxybenzenesulfonate, (6-nonanamidocaproyl)oxybenzenesulfonate, (6-decanamidocaproyl)oxybenzenesulfonate, and mixtures thereof, benzoxazin-type activators, acyl lactam activators (especially acyl caprolactams and acyl valerolactams); and (15) any other known detergent adjunct ingredients, including but not limited to carriers, hydrotropes, processing aids, dyes or pigments (especially hueing dyes), perfumes (including both neat perfumes and perfume microcapsules), and solid fillers.

### Test Methods

### Test 1: Percent Open Cell Content of the Article

The Percent Open Cell Content is measured via gas pycnometry. Gas pycnometry is a common analytical technique that uses a gas displacement method to measure volume accurately. Inert gases, such as helium or nitrogen, are used as the displacement medium. A sample of the flexible, dissolvable, porous article of the present invention is sealed in the instrument compartment of known volume, the appropriate inert gas is admitted, and then expanded into another precision internal volume. The pressure before and after expansion is measured and used to compute the sample article volume.

ASTM Standard Test Method D2856 provides a procedure for determining the percentage of open cells using an older model of an air comparison pycnometer. This device is no longer manufactured. However, one can determine the percentage of open cells conveniently and with precision by performing a test which uses Micromeritics' AccuPyc Pycnometer. The ASTM procedure D2856 describes 5 methods (A, B, C, D, and E) for determining the percent of open cells of foam materials. For these experiments, the samples can be analyzed using an Accupyc 1340 using nitrogen gas with the ASTM foampyc software. Method C of the ASTM procedure is to be used to calculate to percent open cells. This method simply compares the geometric volume as determined using calipers and standard volume calculations to the open cell volume as measured by the Accupyc, according to the following equation:

Percent Open Cell Content (%)=Open cell volume of sample/Geometric volume of sample*100%

It is recommended that these measurements be conducted by Micromeretics Analytical Services, Inc. (One Micromeritics Dr, Suite 200, Norcross, GA 30093). More information on this technique is available on the Micromeretics Analytical Services web sites (www.particletesting.com or www.micromeritics.com), or published in “Analytical Methods in Fine particle Technology” by Clyde Orr and Paul Webb.

Test 2: Micro-Computed Tomographic (gCT) Method for Determining Overall Average Pore Size and Average Cell Wall Thickness of the Open Cell Foams (OCF)

Porosity is the ratio between void-space to the total space occupied by the OCF. Porosity can be calculated from pCT scans by segmenting the void space via thresholding and determining the ratio of void voxels to total voxels. Similarly, solid volume fraction (SVF) is the ratio between solid-space to the total space, and SVF can be calculated as the ratio of occupied voxels to total voxels. Both Porosity and SVF are average scalar-values that do not provide structural information, such as, pore size distribution in the height-direction of the OCF, or the average cell wall thickness of OCF struts.

To characterize the 3D structure of the OCFs, samples are imaged using a pCT X-ray scanning instrument capable of acquiring a dataset at high isotropic spatial resolution. One example of suitable instrumentation is the SCANCO system model 50 pCT scanner (Scanco Medical AG, Brüttisellen, Switzerland) operated with the following settings: energy level of 45 kVp at 133 pA; 3000 projections; 15 mm field of view; 750 ms integration time; an averaging of 5; and a voxel size of 3 μm per pixel. After scanning and subsequent data reconstruction is complete, the scanner system creates a 16 bit data set, referred to as an ISQ file, where grey levels reflect changes in x-ray attenuation, which in turn relates to material density. The ISQ file is then converted to 8 bit using a scaling factor.

Scanned OCF samples are normally prepared by punching a core of approximately 14 mm in diameter. The OCF punch is laid flat on a low-attenuating foam and then mounted in a 15 mm diameter plastic cylindrical tube for scanning. Scans of the samples are acquired such that the entire volume of all the mounted cut sample is included in the dataset. From this larger dataset, a smaller sub-volume of the sample dataset is extracted from the total cross section of the scanned OCF, creating a 3D slab of data, where pores can be qualitatively assessed without edge/boundary effects.

To characterize pore-size distribution in the height-direction, and the strut-size, Local Thickness Map algorithm, or LTM, is implemented on the subvolume dataset. The LTM Method starts with a Euclidean Distance Mapping (EDM) which assigns grey level values equal to the distance each void voxel is from its nearest boundary. Based on the EDM data, the 3D void space representing pores (or the 3D solid space representing struts) is tessellated with spheres sized to match the EDM values. Voxels enclosed by the spheres are assigned the radius value of the largest sphere. In other words, each void voxel (or solid voxel for struts) is assigned the radial value of the largest sphere that that both fits within the void space boundary (or solid space boundary for struts) and includes the assigned voxel.

The 3D labelled sphere distribution output from the LTM data scan can be treated as a stack of two-dimensional images in the height-direction (or Z-direction) and used to estimate the change in sphere diameter from slice to slice as a function of OCF depth. The strut thickness is treated as a 3D dataset and an average value can be assessed for the whole or parts of the subvolume. The calculations and measurements were done using AVIZO Lite (9.2.0) from Thermo Fisher Scientific and MATLAB (R2017a) from Mathworks.

### Test 4: Final Moisture Content

Final moisture content of the sheets of the present invention is obtained by using a Mettler Toledo HX204 Moisture Analyzer (S/N B706673091). A minimum of 1 g of a dried sheet is placed on the measuring tray. The standard program is then executed, with additional program settings of 10 minutes analysis time and a temperature of 110° C.

Test 5: Thickness Thickness of the flexible, porous, dissolvable sheet is obtained by using a micrometer or thickness gage, such as the Mitutoyo Corporation Digital Disk Stand Micrometer Model Number IDS-1012E (Mitutoyo Corporation, 965 Corporate Blvd, Aurora, IL, USA 60504). The micrometer has a 1-inch diameter platen weighing about 32 grams, which measures thickness at an application pressure of about 0.09 psi (6.32 μm/cm²).

The thickness of the flexible, porous, dissolvable sheet is measured by raising the platen, placing a section of the sheet article on the stand beneath the platen, carefully lowering the platen to contact the sheet article, releasing the platen, and measuring the thickness of the sheet in millimeters on the digital readout. The sheet should be fully extended to all edges of the platen to make sure thickness is measured at the lowest possible surface pressure, except for the case of more rigid substrates which are not flat.

### Test 6: Basis Weight

Basis Weight of the flexible, porous, dissolvable sheets of the present invention is calculated as the weight of the sheet per area thereof (grams/m²). The area is calculated as the projected area onto a flat surface perpendicular to the outer edges of the article. The sheets of the present invention are cut into sample squares of 10 cm×10 cm, so the area is known. Each of such sample squares is then weighed, and the resulting weight is then divided by the known area of 100 cm² to determine the corresponding basis weight.

For an object of an irregular shape, if it is a flat object, the area is thus computed based on the area enclosed within the outer perimeter of such object. For a spherical object, the area is thus computed based on the average diameter as 3.14×(diameter/2)². For a cylindrical object, the area is thus computed based on the average diameter and average length as diameter×length. For an irregularly shaped three-dimensional object, the area is computed based on the side with the largest outer dimensions projected onto a flat surface oriented perpendicularly to this side. This can be accomplished by carefully tracing the outer dimensions of the object onto a piece of graph paper with a pencil and then computing the area by approximate counting of the squares and multiplying by the known area of the squares or by taking a picture of the traced area (shaded-in for contrast) including a scale and using image analysis techniques.

### Test 7: Density

Density of the flexible, porous, dissolvable article of the present invention is determined by the equation: Calculated Density=Basis Weight of porous solid/(Porous Solid Thickness×1,000). The Basis Weight and Thickness of the article are determined in accordance with the methodologies described hereinabove.

### Test 8: Specific Surface Area

The Specific Surface Area of the flexible, porous, dissolvable article is measured via a gas adsorption technique. Surface Area is a measure of the exposed surface of a solid sample on the molecular scale. The BET (Brunauer, Emmet, and Teller) theory is the most popular model used to determine the surface area and is based upon gas adsorption isotherms. Gas Adsorption uses physical adsorption and capillary condensation to measure a gas adsorption isotherm. The technique is summarized by the following steps; a sample is placed in a sample tube and is heated under vacuum or flowing gas to remove contamination on the surface of the sample. The sample weight is obtained by subtracting the empty sample tube weight from the combined weight of the degassed sample and the sample tube. The sample tube is then placed on the analysis port and the analysis is started. The first step in the analysis process is to evacuate the sample tube, followed by a measurement of the free space volume in the sample tube using helium gas at liquid nitrogen temperatures. The sample is then evacuated a second time to remove the helium gas. The instrument then begins collecting the adsorption isotherm by dosing krypton gas at user specified intervals until the requested pressure measurements are achieved. Samples may then analyzed using an ASAP 2420 with krypton gas adsorption. It is recommended that these measurements be conducted by Micromeretics Analytical Services, Inc. (One Micromeritics Dr, Suite 200, Norcross, GA 30093). More information on this technique is available on the Micromeretics Analytical Services web sites (www.particletesting.com or www.micromeritics.com), or published in a book, “Analytical Methods in Fine Particle Technology”, by Clyde Orr and Paul Webb.

### EXAMPLES

### Example 1: Comparative Tensile Strength of OCF Sheets with Different PVA Contents

A flexible, dissolvable, and porous sheet containing 57% PVA (“Inventive Example A”) and another flexible, dissolvable, and porous sheet containing 20% PVA (“Comparative Example I”) are prepared using the drum-drying process disclosed in WO2020147000. The final compositions of the dried sheets are as follows:

TABLE 1

| | Wt (%) | | | |
|---|---|---|---|---|
| | Inventive | Comparative | Inventive | Comparative |
| Ingredients | Ex. A | Ex. I | Ex. B | Ex. II |
| Polyvinyl alcohol* | 37% | 20% | 38% | 20% |
| Polyvinyl alcohol** | 20% | — | 21% | — |
| Glycerin | 25% | 9% | 20% | 9% |
| Disodium Cocoyl | 5% | — | — | — |
| Glutamate | | | | |
| Lauramine Oxide | 5% | — | — | — |
| Sodium-Laureth- | — | 47% | 10% | 47% |
| 1-Sulfate | | | | |
| Sodium-Laureth- | — | 12% | 3% | 12% |
| 3-Sulfate | | | | |
| Miscellaneous | — | 4% | — | 4% |
| Water | 8% | 8% | 8% | 8% |

*Commercially available from Changcun with a weight average Mw of 85,000, a degree of polymerization of 1700, and a degree of hydrolysis of 87%.
**Commercially available from Changcun with a weight average Mw of 25,000, a degree of polymerization of 500, and a degree of hydrolysis of 87%.

Following table shows various physical parameters, including the tensile stress, of the resulting Inventive Example A and Comparative Example I.

TABLE 2

| | Inventive | Comparative | Inventive | Comparative |
|---|---|---|---|---|
| | Ex. A | Ex. I | Ex. B | Ex. II |
| Mass (g) | 1.20 | 1.70 | 0.60 | 0.62 |
| Thickness (mm) | 1.22 | 1.30 | 2.00 | 1.65 |
| Cross Sectional | 31 | 33 | 50.8 | 41.8 |
| Area (mm²) | | | | |
| Basis Weight (g/m²) | 315.0 | 446.2 | 157.5 | 163.6 |
| Density (g/L) | 258.2 | 343.2 | 78.7 | 99.4 |
| Tensile | 0.40 | 0.13 | 0.17 | 0.07 |
| Stress (MPa) | | | | |

It is clear from the above data that the high-density Comparative Example I sheet has a significantly lower film strength indicated by a significantly lower tensile stress than that of the high-density Inventive Example A sheet, and that the low-density Comparative Example II sheet has a significantly lower film strength indicated by a significantly lower tensile stress than that of the low-density Inventive Example B sheet. Therefore, inventive examples (of either low or high density) falling within the scope of the present invention have exhibited significantly improved film strength than comparative examples of comparable densities but falling outside of the scope of the present invention.

### Example 2: Comparative Dissolution Rates of OCF Sheets with Different Glycerin Contents

A flexible, dissolvable, and porous sheet containing 15% glycerin and 75% PVA (“Inventive Example C”) and another flexible, dissolvable, and porous sheet containing 2% glycerin and 75% PVA (“Comparative Example III”) are prepared using the hotplate-drying process disclosed in WO2020147000. The final compositions of the dried sheets are as follows:

TABLE 3

Wt (%)

| | Inventive | Comparative |
|---|---|---|
| Ingredients | Ex. C | Ex. III |
| Polyvinyl alcohol* | 75% | 75% |
| Glycerin | 15% | 2% |
| Sodium laureth-3 sulfate | 4% | 17% |
| Water | 6% | 6% |

*Commercially available from Changcun with a weight average Mw of 120,000, a degree of polymerization of 1700, and a degree of hydrolysis of 87%.

Each of the Inventive Example C and Comparative Example III is fully dissolved in deionized water at a sheet-to-water mass ratio of about 0.25. A rheometer is then used to measure the stress and strain response of the sample solution via oscillatory amplitude testing at a frequency of 1 Hz. The strain rate is varied from 0.1 to 1000.0%, in logarithmic steps for a total of 40 measurement points. All 40 data points are used in the calculation of the average values. The resulting average viscosity, average shear modulus, and average phase angle of each sample sheet are recorded as follows:

TABLE 4

| | Inventive | Comparative |
|---|---|---|
| | Ex. C | Ex. III |
| Average viscosity (Pa · s) | 44.3 | 268.9 |
| Average shear modulus | 278.1 | 1689.3 |
| Average phase angle | 78.6 | 62.0 |

The average viscosity and shear modulus are indicative of the gel strength of the respectively dissolved sheet, which inversely correlates with the dissolution rate of the sheet in water. It is clear from the above data that the Comparative Example III sheet has a gel strength that is more than six times of that of the Inventive Example C sheet, which means that the Comparative Example III sheet has a significantly slower dissolution rate in water than that of the Inventive Example C sheet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A flexible, dissolvable, and porous sheet, comprising:
a) from about 50% to about 85% of a water-soluble polymer by total weight of said sheet;
b) from about 1% to about 40% of a surfactant by total weight of said sheet; and
c) from about 10% to about 40% of glycerin by total weight of said sheet,
wherein said flexible, dissolvable, and porous sheet is characterized by a Percent Open Cell Content of from about 80% to about 99% and an Overall Average Pore Size of from about 100 μm to about 2000 μm.

2. The flexible, dissolvable, and porous sheet of claim 1, comprising from about 55% to about 80% of said water-soluble polymer by total weight of said sheet; wherein said water-soluble polymer is selected from the group consisting of polyvinyl alcohols, polyvinylpyrrolidones, polyalkylene oxides, starch and starch derivatives, pullulan, gelatin, methyl celluloses, carboxymethyl celluloses, hydroxypropyl methyl celluloses, and any combinations thereof.

3. The flexible, dissolvable, and porous sheet of claim 1, where said water-soluble polymer comprises a polyvinyl alcohol that is characterized by: (1) a weight average molecular weight of from about 50,000 to about 400,000 Daltons; and (2) a degree of hydrolysis ranging from about 60% to about 99%.

4. The flexible, dissolvable, and porous sheet of claim 3, wherein said water-soluble polymer comprises an additional polyvinyl alcohol that is characterized by: (1) a weight average molecular weight of from about 5,000 to about 100,000 Daltons; and (2) a degree of hydrolysis ranging from about 60% to about 99%; and wherein the weight ratio of said additional polyvinyl alcohol to said polyvinyl alcohol ranges from about 0.1 to about 0.9.

5. The flexible, dissolvable, and porous sheet of claim 1, comprising from about 2% to about 30% of said surfactant by total weight of said sheet; wherein said surfactant is an anionic surfactant comprising C₆-C₂₀ linear alkylbenzene sulphonates, C₆-C₂₀ linear or branched alkylalkoxy sulfates, or any combination thereof.

6. The flexible, dissolvable, and porous sheet of claim 1, comprising from about 12% to about 30% of glycerin by total weight of said sheet.

7. The flexible, dissolvable, and porous sheet of claim 1, wherein said sheet comprises at least one of the following properties:
a Percent Open Cell Content of from 90% to 99%;
an Overall Average Pore Size of from 200 μm to 600 μm;
an Average Cell Wall Thickness of from 10 μm to 80 μm;
a final moisture content of from 3% to 10%, by weight of said sheet;
a thickness of from 0.5 mm to 1.5 mm;
a basis weight of from 35 grams/m² to 200 grams/m²;
a density of from 0.075 grams/cm³ to 0.15 grams/cm³; and
a Specific Surface Area of from 0.1 m²/g to 0.18 m²/g.

8. The flexible, dissolvable, and porous sheet of claim 1, wherein said sheet comprises at least one of the following properties:
a Percent Open Cell Content of from 85% to 99%;
an Overall Average Pore Size of from 150 μm to 1000 μm;
an Average Cell Wall Thickness of from 5 μm to 200 μm;
a final moisture content of from 0.5% to 25%, by weight of said sheet;
a thickness of from 0.3 mm to 4 mm;
a basis weight of from 15 grams/m² to 1000 grams/m²;
a density of from 0.05 grams/cm³ to 0.5 grams/cm³; and
a Specific Surface Area of from 0.03 m²/g to 0.25 m²/g.

9. A unitary detergent article comprising: (a) two or more flexible, dissolvable, and porous sheets of claim 1; and (b) one or more solid dissolvable components located between said two or more sheets, wherein each of said one or more solid dissolvable components comprises a cleansing active.

10. The unitary detergent article of claim 9, wherein said one or more solid dissolvable components comprise particles, pastes, layers, films, sheets, or any combination thereof.

11. The unitary detergent article of claim 9, wherein said one or more solid dissolvable components comprises:
a plurality of discrete particles;
one or more continuous layers of paste;
one or more discontinuous layers of paste;
one or more fibrous sheets;
one or more non-fibrous sheets; or
a combination thereof.

12. The unitary detergent article of claim 9, wherein said cleaning active is selected from the group consisting of fabric care actives, dishwashing actives, hard surface cleaning actives, beauty and/or skin care actives, personal cleansing actives, hair care actives, oral care actives, feminine care actives, baby care actives, and any combinations thereof.
